(19) United States
(12) Patent Application Publication
(10) Pub. No.: US 2015/0239862 A1
(43) Pub. Date: Aug. 27, 2015

(54) PROCESS FOR THE PREPARATION OF VILANTEROL AND INTERMEDIATES THEREOF

(71) Applicant: PERRIGO API LTD., Bnei Brak (IL)

(72) Inventors: Venkata Lakshmi Narasimha Rao Dammalapati, Hyderabad (IN); Hari Krishna Mudduluru, Hyderabad (IN); Ravindra Aduri, Hyderabad (IN)

(21) Appl. No.: 14/427,658

(22) PCT Filed: Sep. 13, 2013
(86) PCT No.: PCT/IN2013/000556

§ 371 Date: Mar. 12, 2015

(30) Foreign Application Priority Data
Sep. 13, 2012 (IN) 3813/CHE/2012

(51) Int. Cl. C07D 319/08; C07C 217/08; C07C 213/00; C07C 51/41; C07C 41/01; C07D 413/04

(52) U.S. Cl. CPC C07D 319/08; C07C 41/01; C07D 413/04; C07C 213/00; C07C 51/412; C07C 217/08

(57) ABSTRACT

An improved process for the preparation of vilanterol and pharmaceutically acceptable salts thereof is disclosed. More specifically the improved process for preparing intermediates for the preparation of vilanterol is disclosed.

### PRIORITY

This application claims the benefit under Indian Provisional Application No. 3813/CHE/2012 filed on 13 Sep. 2012 and entitled “An Improved Process for the preparation of Vilanterol and Intermediates”, the content of which is incorporated by reference herein.

### FIELD OF THE INVENTION

The present invention relates to an improved process for the preparation of Vilanterol or a pharmaceutically acceptable salt thereof and its intermediates in high yield and purity.

### BACKGROUND OF THE INVENTION

Various phenethanolamine derivatives along with the process for their preparation, compositions containing them and their use in medicine, particularly in the prophylaxis and treatment of respiratory diseases were disclosed in PCT publication WO 2003/024439. Vilanterol, being one among the phenethanolamine derivatives is being disclosed in the said PCT application.

Vilanterol is chemically described as 4-{(1R)-2-[6-{2-(2,6-dichlorobenzyl)oxy]ethoxy}hexyl)amino]-1-hydroxyethyl}-2-(hydroxymethyl) phenol as represented by Formula I.

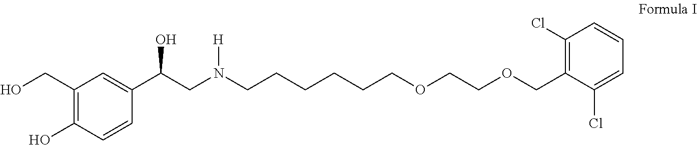 [SMILES: OCc1cc([C@@H](O)CNCCCCCCOCCOCc2c(Cl)cccc2Cl)ccc1O]

The compound 4-{(1R)-2-[(6-{2-[(2,6-dichlorobenzyl)oxy]ethoxy}hexyl)amino]-1-hydroxy ethyl}-2-(hydroxymethyl)phenol is specifically described in WO2003/024439, as are pharmaceutically acceptable salts thereof, in particular the acetate, triphenylacetate, α-phenylcinnamate, 1-naphthoate and (R)-mandelate salts. More specifically the preferred pharmaceutically acceptable salt is triphenylacetate salt.

The PCT publication WO 2003/024439, the corresponding US equivalent U.S. Pat. No. 7,361,787 (herein after the '787 patent) and J. Med. Chem, 2010, 53, 4522-4530 discloses the process for preparation of vilanterol along with pharmaceutically acceptable salt. The '787 patent reaction sequence is schematically represented as follows:

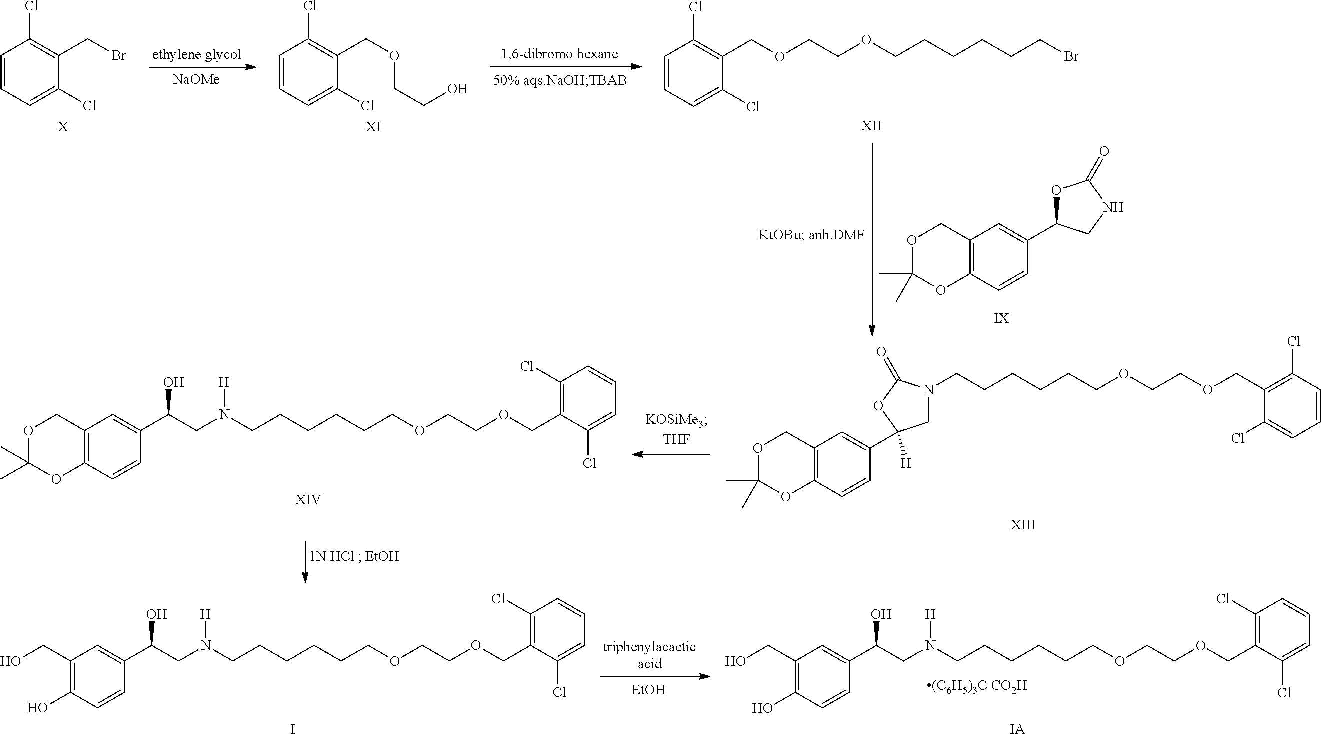

The process described in the '787 patent uses alcoholic solvent during the acetonide cleavage of Formula XIV, which tends to result in the formation of the corresponding ether impurities. This requires repetitive purifications, which can be tedious to practice during scale up process. Moreover the dibromo hexane used in the process contains the corresponding 1,5-dibromo alkanes which tends to react in the same sequential manner to generate the corresponding analogues, which requires repetitive purifications to separate out from the final API. The '787 patent imply the use of column chromatographic procedures which are not feasible on the commercial scale.

The '787 patent further elucidates the process for preparing (5R)-5-(2,2-dimethyl-4H-1,3-benzodioxin-6-yl)-1,3-oxazolidin-2-one (compound IX). The process represented schematically as follows:

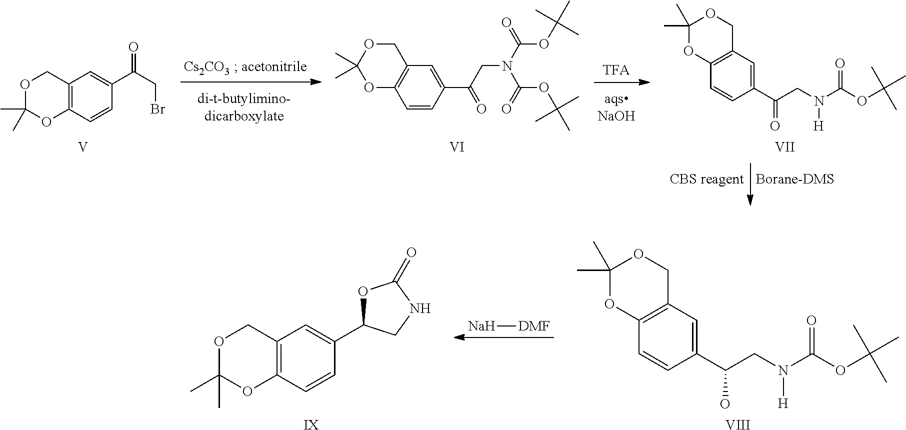

The '787 patent remains silent about the chiral purity of the oxazolidinone compound IX, an essential component which is needed to be chiral pure in order to make the final API free from the corresponding isomeric impurities. The presence of the corresponding isomeric impurities for the chiral intermediate would carry forward during the process which results in the formation of various isomeric impurities which are difficult to separate and need more tedious procedures. Moreover reagents like sodium hydride are difficult to handle during the scale up process as it tends to generate high exothermicity, which can affect the yield and purity of the said compound.

The purity and the yield of vilanterol trifenatate as per the disclosed process are not satisfactory and also the said process involves chromatography techniques to isolate the intermediate compounds. The said techniques are tedious, labor intensive, time consuming process not suitable for industrial scale and which in turn result to an increase in the manufacturing cost. Moreover the said process involves the use of vilanterol trifenatate which degrades to form certain impurities and results in the formation of the final compound with a lesser purity.

In view of intrinsic fragility there is a need in the art to develop a simple, industrially feasible and scalable process for the synthesis of vilanterol that would avoid the aforementioned difficulties. Moreover it becomes necessary to prepare highly chiral pure oxazolidinone intermediate to prepare chirally pure vilanterol.

The present inventors have found an improved process for the preparation of vilanterol by involving either of selection of reaction solvents or precipitation/crystallization techniques, which are useful for the scale up process while retaining the chemical and chiral purity of the product.

### SUMMARY OF THE INVENTION

The present invention encompasses an improved process for the preparation of vilanterol and its pharmaceutically acceptable salts thereof, particularly vilanterol trifenatate in the crystalline form and selective conditions to crystallize substantially pure vilanterol trifenatate such as selection of solvent medium, thereby avoiding the column chromatography process, thus increasing the yield and purity.

In accordance with one embodiment, the present invention provides a process for preparing vilanterol or a pharmaceutically acceptable salt thereof of Formula I;

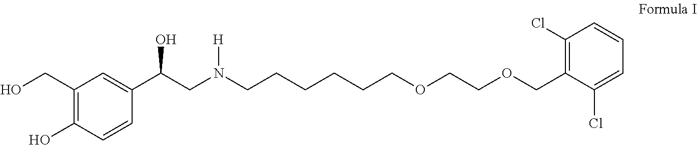 [SMILES: OCc1cc([C@@H](O)CNCCCCCCOCCOCc2c(Cl)cccc2Cl)ccc1O]

comprising:
a) reacting 2,6-dichlorobenzyl halide

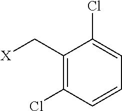

(X is either F or Cl or Br or I); with ethylene glycol in presence of a suitable base to obtain 2-(2,6-dichlorobenzyloxy) ethanol,

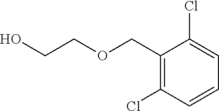 [SMILES: OCCOCc1c(Cl)cccc1Cl]

b) reacting the 2-(2,6-dichlorobenzyloxy) ethanol with 1,6-dihalo hexane of formula

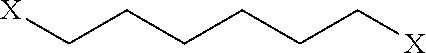

(X is either F or Cl or Br or I); in presence of a base and optionally in the presence of a phase transfer catalyst in a suitable organic solvent to obtain 2-[2-(6-halo-hexyloxy)-ethoxymethyl]-1,3-dichloro-benzene Formula XIIA,

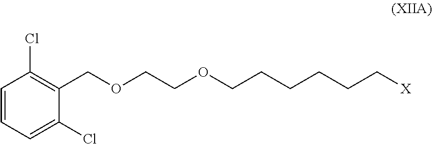

(X is either F or Cl or Br or I)
c) condensing the resultant compound of Formula XIIA with (5R)-5-(2,2-dimethyl-4H-1,3-benzodioxin-6-yl)-1,3-oxazolidin-2-one of Formula IX,

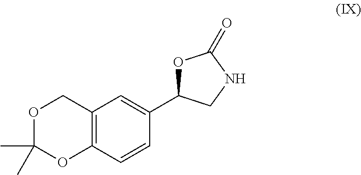 [SMILES: CC1(C)OCc2cc([C@@H]3CNC(=O)O3)ccc2O1]

in presence of a suitable base and a solvent to obtain (5R)-3-{6-[2-(2,6-dichloro benzyloxy)-ethoxy]-hexyl}-5-(2,2-dimethyl-4H-benzo[1,3]dioxin-6-yl)-oxazolidin-2-one

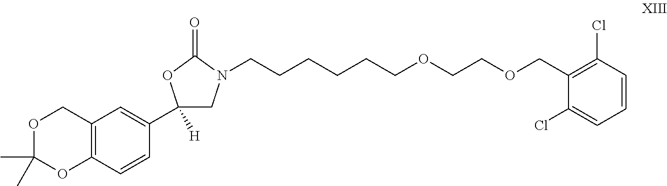 [SMILES: CC1(C)OCc2cc([C@@H]3CN(CCCCCCOCCOCc4c(Cl)cccc4Cl)C(=O)O3)ccc2O1]

d) cleaving the compound of step (c) in presence of a suitable base and a suitable solvent to obtain 4-(1R)-2-[(6-{2-[(2,6-dichlorobenzyl)oxy]ethoxy}hexyl)amino]-1-(2,2-di methyl-4H-1,3-benzodioxin-6-yl) ethanol of Formula XIV,

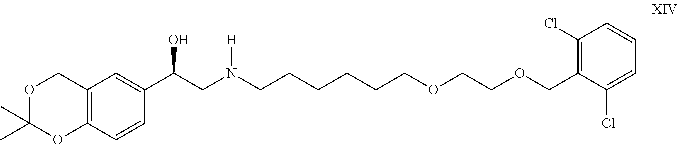 [SMILES: CC1(C)OCc2cc([C@@H](O)CNCCCCCCOCCOCc3c(Cl)cccc3Cl)ccc2O1]

e) deprotecting the compound of step (d) in presence of an acid and a suitable nonalcoholic solvent to obtain substantially pure vilanterol,
f) optionally converting vilanterol into a pharmaceutically acceptable salt of vilanterol in a suitable nonalcoholic solvent medium.

In accordance with a second embodiment, the present invention provides a process for preparing vilanterol or a pharmaceutically acceptable salt thereof, comprising:
a) condensing 2-(2,6-dichlorobenzyloxy) ethanol with 1,6-dihalo hexane of formula

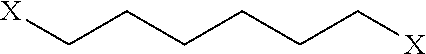

(X is either F or Cl or Br or I)
in presence of a base, in a suitable organic solvent and optionally in presence of a phase transfer catalyst, to obtain 2-[2-(6-halo-hexyloxy)-ethoxymethyl]-1,3-dichloro-benzene Formula XIIA,

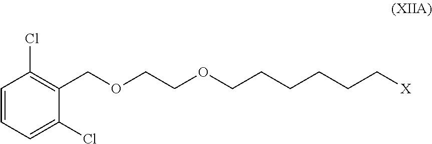

(X is either F or Cl or Br or I)
   wherein the 1,6-dihalo hexane comprises less than 0.15% of corresponding dihalo alkane impurities; and
b) converting the compound of Formula XIIA into vilanterol or a pharmaceutically acceptable salt thereof.

In accordance with a third embodiment, the present invention provides a process for preparing vilanterol or a pharmaceutically acceptable salt thereof comprising:
a) condensing a compound of Formula XIIA

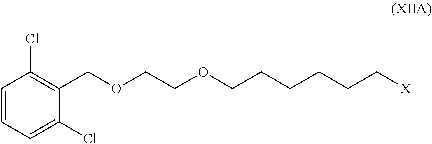

(X is either F or Cl or Br or I)
   with (5R)-5-(2,2-dimethyl-4H-1,3-benzodioxin-6-yl)-1,3-oxazolidin-2-one of Formula IX,

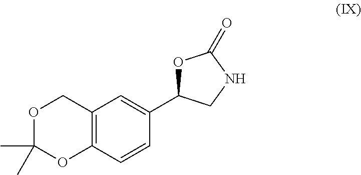 [SMILES: CC1(C)OCc2cc([C@@H]3CNC(=O)O3)ccc2O1]

in presence of a suitable base and a solvent to obtain substantially pure (5R)-3-{6-[2-(2,6-dichloro benzyloxy)-ethoxy]-hexyl}-5-(2,2-dimethyl-4H-benzo[1,3]dioxin-6-yl)-oxazolidin-2-one of Formula XIII,

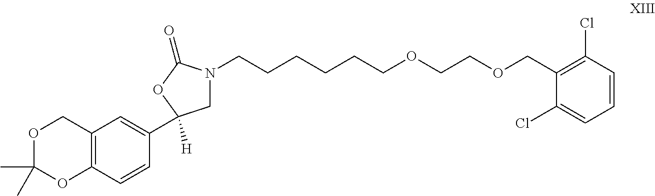 [SMILES: CC1(C)OCc2cc([C@@H]3CN(CCCCCCOCCOCc4c(Cl)cccc4Cl)C(=O)O3)ccc2O1]

wherein the said oxazolidinone compound of Formula IX is substantially free from the (S)— isomer.
b) converting the compound obtained in step (a) to vilanterol or a pharmaceutically acceptable salt thereof.

In accordance with a fourth embodiment, the present invention provides a process for preparing vilanterol or a pharmaceutically acceptable salt thereof, comprising,
a) deprotecting 4-(1R)-2-[(6-{2-[(2,6-dichloro benzyl)oxy]ethoxy}hexyl)amino]-1-(2,2-di methyl-4H-1,3-benzodioxin-6-yl)ethanol of Formula XIV

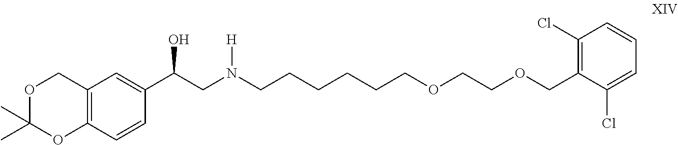 [SMILES: CC1(C)OCc2cc([C@@H](O)CNCCCCCCOCCOCc3c(Cl)cccc3Cl)ccc2O1]

with a suitable acid in presence of a nonalcoholic solvents to obtain vilanterol,
b) optionally converting the vilanterol into a pharmaceutically acceptable salt of vilanterol in a suitable nonalcoholic solvent medium.

In accordance with a fifth embodiment, the present invention provides a process for preparing vilanterol or a pharmaceutically acceptable salt thereof, comprising preparing substantially pure chiral oxazolidinone intermediate of Formula IX,

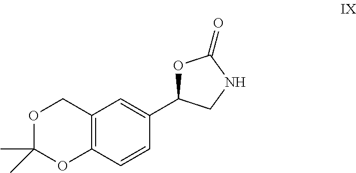 [SMILES: CC1(C)OCc2cc([C@@H]3CNC(=O)O3)ccc2O1]

comprising:
i) reaction of 2-bromo-1-(2,2-dimethyl-4H-1,3-benzodioxin-6-yl) ethanone of Formula V with di-tert-butyl iminodicarboxylate

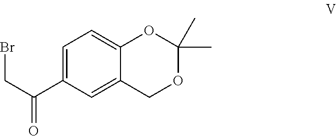 [SMILES: CC1(C)OCc2cc(C(=O)CBr)ccc2O1]

in the presence of a suitable base and an aprotic solvent to obtain di(tert-butyl) 2-(2,2-dimethyl-4H-1,3-benzodioxin-6-yl)-2-oxoethyl imidodicarbonate of Formula VI,

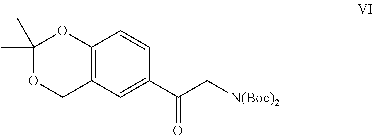

ii) deprotecting the resultant compound from step (i) in the presence of acid to obtain tert-butyl 2-(2,2-dimethyl-4H-1,3-benzodioxin-6-yl)-2-oxoethylcarbamate of Formula VII,

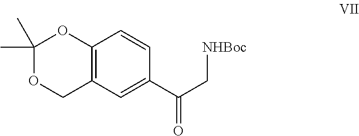 [SMILES: CC(C)(C)OC(=O)NCC(=O)c1ccc2c(c1)COC(C)(C)O2]

iii) reducing the compound of step (ii) with a suitable reducing agent in the presence of suitable chiral auxiliary to obtain substantially pure tert-butyl (2R)-2-(2,2-dimethyl-4H-1,3-benzodioxin-6-yl)-2-hydroxyethyl carbamate of Formula VIII,

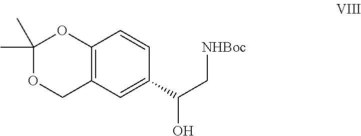

iv) cyclisation of compound of step (iii) in presence of a suitable base and a solvent to obtain substantially pure (5R)-5-(2,2-Dimethyl-4H-1,3-benzodioxin-6-yl)-1,3-oxazolidin-2-one, and
v) conversion of substantially pure (5R)-5-(2,2-Dimethyl-4H-1,3-benzodioxin-6-yl)-1,3-oxazolidin-2-one to vilanterol or pharmaceutically acceptable salts thereof.

In accordance with a sixth embodiment, vilanterol or a pharmaceutically acceptable salt as obtained by the said procedure as described above have a total purity greater than 98%, preferably greater than 99%; more preferably greater than 99.5% as measured by HPLC.

In accordance with a seventh embodiment, the present invention provides vilanterol or a pharmaceutically acceptable salt thereof and its chiral intermediates prepared as per the present invention, having a chiral purity greater than 98% by HPLC; preferably 99%; more preferably 99.5%.

In accordance with an eighth embodiment, the present invention provides 1,6-dibromo hexane used for the preparation of vilanterol comprises less, than 0.15% of corresponding 1,5-dibromo pentane impurity, more preferably less than 0.1%.

In accordance with a ninth embodiment, the present invention provides vilanterol or a pharmaceutically acceptable salt thereof prepared as per the present invention containing less than 0.15% of any impurity, more preferably less than 0.1% as by HPLC.

In accordance with a tenth embodiment, the present invention provides highly chiral pure oxazolidinone compound of formula IX, wherein the R-isomer is greater than 99.8% chiral pure.

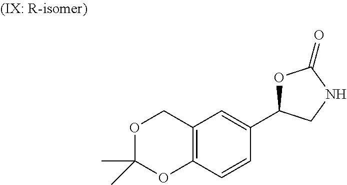 [SMILES: CC1(C)OCc2cc([C@@H]3CNC(=O)O3)ccc2O1]

In accordance with an eleventh embodiment, the present invention provides a pharmaceutical composition comprising vilanterol or a pharmaceutically acceptable salt thereof prepared by the processes of the present invention and at least one pharmaceutically acceptable excipient.

### DETAILED DESCRIPTION OF THE INVENTION

The main objective of the present invention is to provide an industrially feasible process for highly pure vilanterol or a pharmaceutically acceptable salt thereof. The inventors focused majorly on the process modifications which are feasible for scale up process.

In accordance with one embodiment, the present invention provides a process for preparing vilanterol or its pharmaceutically acceptable salt of Formula I;

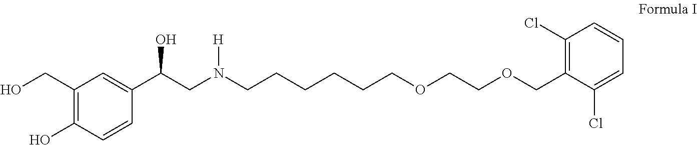 [SMILES: OCc1cc([C@@H](O)CNCCCCCCOCCOCc2c(Cl)cccc2Cl)ccc1O]

comprising:
a) reacting 2,6-dichlorobenzyl halide

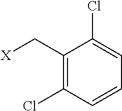

(X is either F or Cl or Br or I), preferably Br; with ethylene glycol in presence of a suitable base to obtain 2-(2,6-dichlorobenzyloxy) ethanol,
b) reacting the 2-(2,6-dichlorobenzyloxy) ethanol with 1,6-dihalo hexane

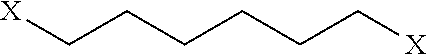

(X is either F or Cl or Br or I), preferably Br; in presence of a base and optionally in the presence of a phase transfer catalyst in a suitable organic solvent to obtain 2-[2-(6-halo-hexyloxy)-ethoxymethyl]-1,3-dichloro-benzene Formula XIIA,

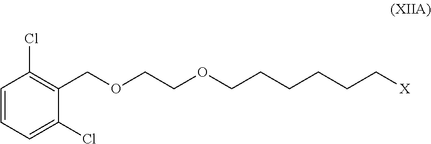

(X is either F or Cl or Br or I)
c) condensing the resultant compound of Formula XIIA with (5R)-5-(2,2-dimethyl-4H-1,3-benzodioxin-6-yl)-1,3-oxazolidin-2-one of Formula IX,

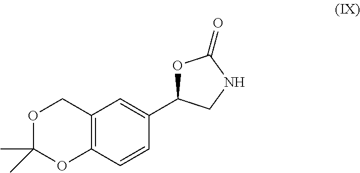 [SMILES: CC1(C)OCc2cc([C@@H]3CNC(=O)O3)ccc2O1]

in presence of a suitable base and a solvent to obtain (5R)-3-{6-[2-(2,6-dichloro benzyloxy)-ethoxy]-hexyl}-5-(2,2-dimethyl-4H-benzo[1,3]dioxin-6-yl)-oxazolidin-2-one
d) cleaving the compound of step (c) in presence of a suitable base and a suitable solvent to obtain 4-(1R)-2-[(6-{2-[(2,6-dichlorobenzyl)oxy]ethoxy}hexyl)amino]-1-(2,2-di methyl-4H-1,3-benzodioxin-6-yl) ethanol
e) deprotecting the compound of step (d) in presence of an acid and a suitable nonalcoholic solvent to obtain substantially pure vilanterol,
f) optionally converting vilanterol into a pharmaceutically acceptable salt of vilanterol in a suitable nonalcoholic solvent medium.

The dihaloalkane derivatives include dihalopentane, dihaloheptane and the like; preferably dihalopentane and the halo refer to bromo.

As mentioned in above process, in step (a) the preferred base is selected from either inorganic or organic base, preferably inorganic bases like alkali metal hydroxides such as lithium hydroxide, sodium hydroxide, potassium hydroxide; alkali metal hydrides like sodium hydride, potassium hydride; alkali metal alkoxides such as sodium methoxide, sodium ethoxide, sodium tert-butoxide, potassium tert-butoxide. More preferably the suitable base is potassium tert-butoxide.

The prior art process implies ethyl acetate, a polar solvent for isolating the compound in step (a) wherein 2,6-dichloro benzyl alcohol and unreacted ethylene glycol are carried further which are difficult to separate. These compounds further may react with the later stage intermediate to generate more impurities. In order to overcome these difficulties the inventors have surprisingly found that, the use of nonpolar solvent can eliminate these impurities at step a) itself. For instance the unreacted compounds as said above may react with compound XII to obtain the following possible impurities.

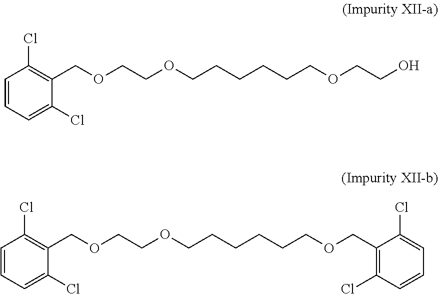 [SMILES: OCCOCCCCCCOCCOCc1c(Cl)cccc1Cl ; Clc1cccc(Cl)c1COCCCCCCOCCOCc1c(Cl)cccc1Cl]

The suitable solvent for isolating the product of step (a) is preferably selected from group of nonpolar solvents ranging from hexane, heptane, cyclohexane, toluene, xylene, chlorobenzene and the like; more preferably toluene.

The dihalo alkane used herein the step (b) is 1,6-dihalo hexane wherein the halo group is selected from F, Cl, Br or I; preferably 1,6-dibromo hexane is used, comprising less than 0.15% of corresponding dihaloalkane derivative. The preferable base in step (b) is selected from inorganic bases like lithium hydroxide, sodium hydroxide and potassium hydroxide; preferably sodium hydroxide. The step b) reaction is carried optionally in the presence of a phase transfer catalyst such as tetrabutylammonium salt; preferably tetra butyl ammonium chloride or bromide or iodide.

The preferable base and the solvent used in step (c) are selected from the aforementioned components, more preferably selected from alkali metal hydrides such as sodium hydride, potassium hydride; alkali metal alkoxides such as sodium methoxide, sodium ethoxide, sodium tert-butoxide, potassium tert-butoxide; more preferably potassium t-butoxide and polar aprotic solvents such as dimethyl formamide (DMF), dimethyl acetamide (DMAc), dimethyl sulfoxide (DMSO), n-methyl-pyrrolidin-2-one (NMP) and the like; preferably dimethyl formamide.

The chiral oxazolidinone intermediate (IX) used herein the condensation step (c) is highly chiral pure. The chiral pure oxazolidinone is prepared by the improved process as mentioned in the foregoing application.

The step d) of the aforementioned process, cleaving the resultant compound of Formula XIII obtained from step c) with a suitable base and a suitable solvent. The suitable base includes, a mild base selected form sodium or potassium trialkyl silanolate, preferably potassium trimethyl silanolate. The solvent used in this step is selected from group of aprotic solvents such as THF, dioxane, DMF, DMAc, DMSO and the like; preferably THF.

The step e) of the aforementioned process, the deprotection of the acetonide group in compound XIV is carried out in the presence of an acid and a nonalcoholic solvent to obtain vilanterol in high purity. The suitable acid includes, but is not limited to HCl, HBr or their aqueous components, HCl dissolved in suitable solvents in the likes of DMF-HCl, EtOAc—HCl, ether-HCl, dioxane-HCl and the like; preferably aqueous HCl.

The preferred nonalcoholic solvent may be selected from the group consisting of halogenated solvents, esters, ethers, hydrocarbons, amides, ketones and nitriles or mixtures thereof. Preferably the nonalcoholic solvent is selected from methylene chloride, ethyl acetate, THF, DMF, acetone, acetonitrile and the like; more preferably acetone.

The inventors have found the influence of solvent system in the final purity of vilanterol. The inventors have found that the solvent system plays an important role in maintaining the impurities below par and also retention of desired configuration reducing the racemization process. A pharmaceutically acceptable component with chiral nature should be highly chiral pure in order to design the final API meeting the regulatory guidelines. During optimization studies, acetonide deprotection was carried out as per reported process in US '787 patent using EtOH (6.0 V)/1NHCl (6.0 V)/RT/1 h and observed around 95-96% purity by HPLC at crude stage. After column purification using 10% EtOH in DCM/1.0% ammonia, observed marginal improvement in purity and formation of new impurities at 1.24 and 1.26 RRTs (possibly degradants). In reported condition, impurities formation was observed at 1.17 and 1.19 RRT which are increased during prolonged maintenance (we deduced them as possible ethyl ethers impurities). Thus the use of alcoholic solvents were eliminated in this stage.

Further, reactions were carried out in THF (5.0 V)/1N HCl (6.0 V)/RT and observed better chemical purities (98-99%) compared to reported condition. In this condition, racemization was higher side compared to reported condition. Following studies were carried out to check racemization on prolonged maintenance.

| Condition | Initial S-Isomer | After 4 h | After 12 h |
|---|---|---|---|
| THF/1N HCl/RT | 0.34% | Not checked | 6.0% |
| EtOH/1N HCl/RT | 0.15% | 0.2% | 2.21% |
| THF/1N HCl/RT | 0.15% | Not checked | 6.36% |

Acetonide deprotection was tried in acetone (10 V) using 0.5 N HCl (12 V) at RT for 2-3 h and observed starting material content around 1.5% by HPLC. Further progress was not observed and impurities formation less compared to all other conditions. To remove unreacted starting material, column purification was developed using MeOH/DCM and achieved desired quality of the product (>99.0%) with 80-85% yields. In this condition, racemisation was not observed even prolonged hours up to 5 h. This condition was found to be suitable in developing the desired vilanterol, wherein the consistent yields and purity profile was achieved.

The inventors also designed few other conditions for acetonide deprotection. The results are summarised as tabulated herein below:

| | | Yields/ | |
|---|---|---|---|
| S. | | HPLC | |
| No | Conditions | purity | Remarks |
| 1 | Ethanol/1NHCl/ | 75%; | Impurities observed at 1.17, 1.19 |
| | RT/2-3 h | 96.2% | RRTs which cannot be eliminated at |
| | | | final step |
| | | | Product degradation observed and |
| | | | racemization observed prolonged |
| | | | maintenance |
| 2 | THF/1NHCl/ | 80%; | Chemical purities are better but |
| | RT/2-3 h | 98.9% | racemisation observed |
| 3 | Ethanol/0.5NHCl/ | 90%; | Impurities observed at 1.17, 1.19 |
| | RT/2-3 h | 97.65% | RRTs which cannot be eliminated at |
| | | | final step |
| 4 | 2-methyl THF/1N | 90%; | Racemisation is high in this |
| | HCl/RT/8 h | 95.5% | condition |
| | | | More impurities formation |
| 5 | 2-methoxy ethanol/ | — | Reaction is very slow and impurities |
| | 0.5N HCl/RT/8 h | 88.6% | formation observed |
| 6 | Trifluoro acetic | 87.0%; | More impurities observed |
| | acid/DCM/ | 76.8% | |
| | H₂O/RT/2-3 h | | |
| 7 | PTSA/acetonitrile/ | 90%; | More impurities observed |
| | H₂O/RT/2-3 h | 35.38% | |
| 8 | Amberlyst-15/ | — | No progress was observed |
| | EtOH/RT/12 h | | |
| 9 | Boric acid/EtOH | — | No progress was observed |
| | (95%)/RT-reflux | | |
| 10 | Acetone/0.5N HCl/ | 77%; | Good yields and quality observed |
| | RT/2-3 h | 99.15% | after column purification in |
| | | | MeOH/DCM |

Vilanterol thus obtained as in the above process from steps (a) to (e), is optionally converted in to pharmaceutically acceptable salt as in step (f), wherein the process comprises reacting substantially pure vilanterol (with the undesired S-isomer less than 0.15%) with a suitable acid in the presence of a nonalcoholic solvent.

The pharmaceutically acceptable acid addition salts as used herein include those formed from hydrochloric, hydrobromic, sulphuric, citric, tartaric, phosphoric, lactic, pyruvic, acetic, trifluoroacetic, triphenylacetic, phenylacetic, substituted phenyl acetic acids, succinic, oxalic, fumaric, maleic, malic, glutamic, aspartic, oxaloacetic, methanesulphonic, ethanesulphonic, arylsulponic (for example p-toluenesulphonic, benzenesulphonic, naphthalenesulphonic), salicylic, glutaric, mandelic, cinnamic, substituted cinnamic (for example, methyl, methoxy, halo or phenyl substituted cinnamic, including 4-methyl and 4-methoxycinnamic acid and α-phenyl cinnamic acid), ascorbic acids; preferably triphenylacetic acid.

In another embodiment, the present invention provides vilanterol as obtained by the above said process is converted into pharmaceutically acceptable salt by reacting with a suitable organic or inorganic acids or bases. Preferably acid addition salts are defined as above. Advantageously the preferred acid addition salt is prepared using triphenyl acetic acid.

During optimization studies, salt formation with triphenylacetic acid was carried out as per reported condition in US'787 and observed an impurity on a higher side at 1.24 RRT. This impurity makes the final API combination not suitable for preparing the formulation, which demands a higher chemical and chiral pure. Further the reactions were performed in different alcoholic solvent such as methanol and isopropanol for the formation of desired salt which did not result in exceptional yields and also purity. The role of solvent system is has a major influence on the purity of the compound.

The preferred nonalcoholic solvent may be selected from the group consisting of halogenated solvents, esters, ethers, hydrocarbons, amides, ketones and nitriles or mixtures thereof. Preferably the nonalcoholic solvent is selected from methylene chloride, ethyl acetate, THF, DMF, acetone, acetonitrile and the like; more preferably acetone.

Apart from above said alcoholic solvents, inventors herein, tried with ketone solvents such as acetone, methyl isobutyl ketone considering the results obtained in stage wherein the vilanterol was obtained (prefinal stage). Reactions in acetone resulted in better yields with a decreased impurity profile with greater than 99.5% purity by HPLC, greater than 99.9% chiral pure and no single major impurity >0.1%.

Any conditions for the formation of acid addition salts of vilanterol or the free base form from solution may be used wherein acid addition salts of vilanterol formed, for example concentrated by subjecting the solution to heating, cooling the solution to precipitation, crystallization, solvent precipitation, drying and the like.

In another embodiment, the present invention provides a process for preparing the substantially pure chiral oxazolidinone intermediate of Formula IX, comprising:
i) reaction of 2-bromo-1-(2,2-dimethyl-4H-1,3-benzodioxin-6-yl) ethanone of Formula V with di-tert-butyl iminodicarboxylate in the presence of a suitable base and an aprotic solvent to obtain di(tert-butyl) 2-(2,2-dimethyl-4H-1,3-benzodioxin-6-yl)-2-oxoethyl imidodicarbonate of Formula VI,
ii) deprotecting the resultant compound from step (i) in the presence of acid to obtain tert-butyl 2-(2,2-dimethyl-4H-1,3-benzodioxin-6-yl)-2-oxoethylcarbamate of Formula VII,
iii) reducing the compound of step (ii) with a suitable reducing agent in the presence of suitable chiral auxiliary to obtain substantially pure tert-butyl (2R)-2-(2,2-dimethyl-4H-1,3-benzodioxin-6-yl)-2-hydroxyethyl carbamate of Formula VIII,
iv) cyclisation of compound of step (iii) in presence of a suitable base and solvent to obtain substantially pure (5R)-5-(2,2-Dimethyl-4H-1,3-benzodioxin-6-yl)-1,3-oxazolidin-2-one (IX), wherein the said chiral oxazolidinone of Formula IX, and
v) conversion of substantially pure (5R)-5-(2,2-Dimethyl-4H-1,3-benzodioxin-6-yl)-1,3-oxazolidin-2-one to vilanterol or pharmaceutically acceptable salts.

As mentioned herein the embodiment, the process from steps (i) to (iv), includes the intermediates/products isolated from the reaction mixture by suitable techniques other than column chromatography including adopting various process modifications such as purification by washings, filtrations, trituration, precipitation crystallization, evaporation etc.

The suitable solvent includes but is not limited to alcohols, esters, ketones, amides, nitriles, ethers, halogenated hydrocarbons, aromatic hydrocarbons, hydrocarbons, water and mixtures thereof.

As mentioned herein in the above process, the step (i) the suitable base as defined above, preferably selected from alkali metal carbonates such as sodium or potassium or cesium carbonates are used. The aprotic solvent used herein the step (i) is preferably acetonitrile, tetrahydrofuran, dioxane, DMF, DMAC, DMSO, NMP and the like, more preferably acetonitrile.

As disclosed herein the resultant compound of formula (VI) was isolated in a two-step process and eliminating column chromatography. The said isolation may involve the process by purification by washings, filtrations, trituration, crystallization, evaporation etc. The first step of isolation involves the use of a preferable solvent source such as ester solvents including methyl acetate, ethyl acetate and the like; halogenated solvents such as dichloromethane, chloroform, chlorobenzene and the like; ether solvents such as diethyl ether methyl ter. butyl ether, diisopropyl ether and the like; aromatic hydrocarbon solvents such as toluene, xylenes and the like. Preferably aromatic hydrocarbon solvents, more preferably toluene. The second step of isolation of compound (VI) involves slurring the product obtained from first step with use of suitable solvents such as esters such as ethyl acetate and the like; ethers such as THF, methyl tert-butyl ether, diisopropyl ether and the like; halogenated hydrocarbons such as methylene chloride and the like; alcoholic solvents such as methanol, ethanol isopropanol and the like; aromatic hydrocarbons such as toluene, xylene and the like and mixtures thereof; more preferably diisopropyl ether.

As mentioned herein the step (ii), the deprotection of compound VI was carried in an acid medium and a suitable organic solvent medium. The preferred acid medium is HCl, HBr or trifluoroacetic acid. The suitable organic solvent for this step is preferably selected from dichloromethane, chloroform, acetonitrile, ethyl acetate, toluene, DMF, water etc. More preferably dichloromethane is used.

As disclosed herein the resultant compound of formula (VII) was isolated by purification involving by washings, filtrations, trituration, crystallization, and evaporation etc thereby eliminating column chromatography. The preferably mode of isolating the desired compound by trituration involves a preferable organic solvent or a mixture thereof, wherein the suitable solvent is as defined above. More preferably the solvent combination is chosen from ether-hydrocarbon solvent mixture including but not limited to diethyl ether-hexane, diethyl ether-heptane, diisopropylether-hexane, diisopropylether-heptane and the like, more preferably diisopropylether-heptane.

As mentioned herein the process of step (iii), the reduction of compound VII is carried in the presence of suitable reducing agent optionally in the presence of a suitable chiral auxiliary. The preferred reducing agent is selected from but not limited to borane reagent complexes such as BH₃-THF, BH₃-DMS, BH₃-pyridine, BH₃-diethylaniline, BH₃-1,4-thioxane, 9-BBN, catechol borane, thexyl borane, disamyl borane, alpine borane, diisopinocamphenyl borane and the like. The chiral auxiliary is selected but not limited to pseudoephedrine, chiral ligands such as BINOL, BINAP, DuPhos; chiral oxazolidinones such as CBS catalysts including (S)-MeCBS, (R)-MeCBS and like. The preferable reducing agent is BH₃-DMS and the chiral auxiliary is (R)-MeCBS.

In an embodiment, the compound of Formula VIII can be isolated from the reaction mixture by,

ia) a suitable organic solvent for extraction from the reaction mixture, followed by concentrating in vacuo to obtain the residue,
iia) optionally treating the residue with a suitable non polar solvent followed by filtration and dried to obtain crude VIII,
iiia) purifying the resultant obtained in step (ia) or (iia) by dissolving in a suitable organic solvent followed by treating with an antisovlent.

The compound of Formula VIII was isolated from the reaction mixture by extracting the desired product into suitable organic solvent such as ethyl acetate or DCM, more preferably ethyl acetate. The nonpolar solvent as mentioned in step (iia) above is preferably selected but not limited to hydrocarbon solvents such as pentane, hexane, heptane, toluene, xylene, chloro benzene and the like. More preferably hexane/heptane is used. As mentioned in step (iiia), the process involves dissolving the crude compound of Formula VIII in a suitable solvent such as acetone or MIBK, preferably acetone. The preferred anti solvent is selected from the group consisting of hydrocarbons such as pentane, hexane, heptane, toluene, xylenes and the like, ethers such as dimethyl ether, diethyl ether, diisopropyl ether, MTBE and the like; water or mixtures thereof; preferably water.

As mentioned herein the step (iv), the cyclisation is carried out in presence of a suitable base and a suitable solvent. The base is selected from the group consisting of alkali metal alkoxides, alkali metal hydroxides, alkali metal carbonates, alkali metal bicarbonates and the like. Preferably the base is selected from the group consisting of sodium methoxide, potassium tert-butoxide, sodium carbonate, potassium carbonate, sodium bicarbonate, potassium bicarbonate and the like; more preferably potassium tert-butoxide. The suitable solvent preferably is DMF.

As mentioned, this chiral oxazolidinone in step (iv) was isolated from the reaction mixture using specific purification techniques such as precipitation, crystallizations, washings and the like. More preferably the compound was obtained by precipitation method. The prior art process in US'787 remains silent about the purification and also the chemical/chiral purity of the said compound. However, the present inventors found erroneous results upon repeating the prior art process with respect to the purity of the compound. The prior art process involves treatment of the reaction mixture with aqueous acid preferably aqs.HCl after completing the reaction and extracting with ethyl acetate to obtain the resultant compound. The inventors found a chance of racemization by treatment of reaction mass in acidic medium. Thus altering the work up procedures resulted in formation of desired compound without racemization.

In another embodiment, the compound of Formula IX may be purified by:
a) dissolving the crude compound in a suitable solvent
b) adding suitable anti solvent to precipitation,
c) filtration to obtain substantially pure desired isomer.
   wherein the suitable solvents and anti-solvent as cited herein are described aforementioned above and optionally repeating the steps (b) to (d) to obtain highly pure R-isomer.

The suitable solvent for dissolving the compound is polar aprotic solvents as defined above, the preferable solvent is DMF and the suitable anti solvent is water.

In another embodiment, the present invention provides (5R)-5-(2,2-dimethyl-4H-1,3-benzodioxin-6-yl)-1,3-oxazolidin-2-one of Formula IX having less than about 0.2% of its corresponding (S)-isomer; preferably less than 0.1% by HPLC.

In another embodiment, the starting material 2-bromo-1-(2,2-dimethyl-4H-1,3-benzodioxin-6-yl) ethanone compound of formula V used in the present invention is prepared from the any known methods in the prior arts US2004/167167 and in US2010/009950 with certain process modifications by eliminating column chromatography and by isolating the said compounds by using crystallization methods. The process comprises;
1) treating 5-bromo-2-hydroxy benzyl alcohol in a suitable solvent or mixture in the presence of Lewis acid catalyst to obtain the corresponding acetonide,
2) acylation of above obtained acetonide with a suitable acylating reagent in the presence of a suitable base, isolating the resultant from solvent or mixture thereof,
3) treating the compound from step (2) by a suitable base, silylating agent and a brominating agent in a suitable solvent, isolating the resultant from a suitable solvent or mixture thereof.
   wherein the suitable solvents, base in the above said steps are aforementioned above.

As forementioned above, the Lewis acid in step (1) is selected from but not limited to, such as aluminum chloride, zinc chloride, aluminium bromide and the like. More preferably aluminum chloride or zinc chloride. The suitable solvent in step (a) is selected but not limits to foresaid solvent. More preferably acetone-THF.

As forementioned above, the acylating agent in step (2) is selected from but are not limited to acetyl chloride, acetyl bromide, acetic anhydride, wenierbamide such as N-methoxy —N-methylacetamide More preferably N-methoxy —N-methylacetamide. The suitable base in step (2) is preferably n-BuLi. The suitable solvent for isolating the compound from step (2) is diisopropylether-heptane.

The suitable base in step (3) is from sodium bis(trimethyl silylamide) and the silylating agent is sleeted from trimethylsilyl halide such as trimethylsilyl chloride, trimethylsilyl bromide, trimethylsilyl iodide; more preferably trimethylsilyl chloride. The suitable brominating agent is preferably selected from but are not limited to bromine solution, trimethyl silyl bromide, HBr in acetic acid, HBr in water and the like; more preferably bromine solution. The suitable solvent for isolating the desired compound is heptane.

In another embodiment, vilanterol or pharmaceutically acceptable salt as obtained by the aforementioned process as described above is having a total chemical purity greater than 98%, preferably greater than 99%; more preferably greater than 99.5% as measured by HPLC.

In another embodiment, vilanterol or a pharmaceutically acceptable salt thereof and its chiral intermediates prepared as per the present invention, having a chiral purity greater than 98% by HPLC; preferably 99%; more preferably 99.5%.

In another embodiment, vilanterol or a pharmaceutically acceptable salt thereof prepared as per the present invention substantially containing less than 0.15% of any impurity, more preferably less than 0.1% as by HPLC. The possible impurities during the process for preparation of vilanterol according to the invention are tabulated below in Table 1.

| S. No | Impurity | Structure | Description |
|---|---|---|---|
| 1 | A | 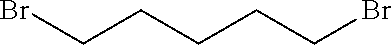 | 1,5-dibromo pentane |
| 2 | B | 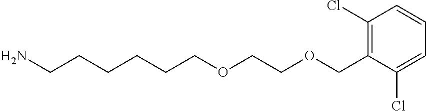 [SMILES: NCCCCCCOCCOCc1c(Cl)cccc1Cl] | Amino ether impurity |
| 3 | C | Degraded impurity | 1.24 RRT |
| 4 | D | 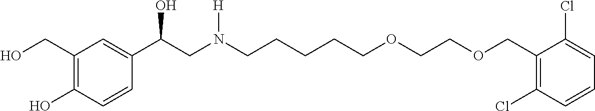 [SMILES: OCc1cc([C@@H](O)CNCCCCCOCCOCc2c(Cl)cccc2Cl)ccc1O] | (R)-isomer of vilanterol analogue |
| 5 | E | 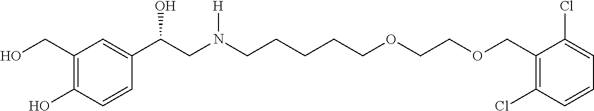 [SMILES: OCc1cc([C@H](O)CNCCCCCOCCOCc2c(Cl)cccc2Cl)ccc1O] | (S)-isomer of vilanterol analogue |
| 6 | F | 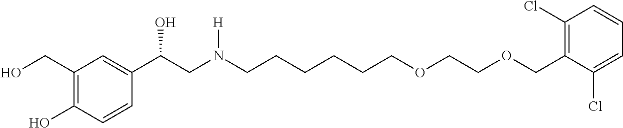 [SMILES: OCc1cc([C@H](O)CNCCCCCCOCCOCc2c(Cl)cccc2Cl)ccc1O] | (S)-vilanterol |

Possible impurities by the process described in US'787 (Table-2)

| S. No | Impurity | Structure | Description |
|---|---|---|---|
| 1 | G | 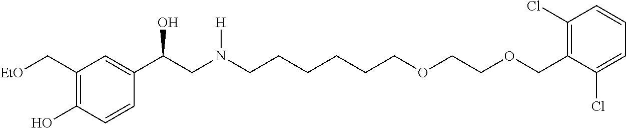 [SMILES: CCOCc1cc([C@@H](O)CNCCCCCCOCCOCc2c(Cl)cccc2Cl)ccc1O] | Monoalkyl derivative |
| 2 | H | 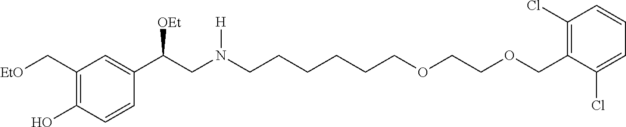 [SMILES: CCOCc1cc([C@H](CNCCCCCCOCCOCc2c(Cl)cccc2Cl)OCC)ccc1O] | Dialkyl derivative |
| 3 | I | 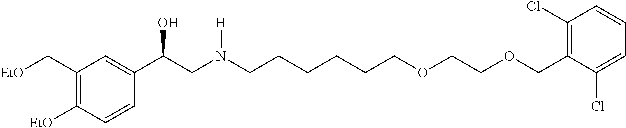 [SMILES: CCOCc1cc([C@@H](O)CNCCCCCCOCCOCc2c(Cl)cccc2Cl)ccc1OCC] | Aromatic dialkyl derivative |
| 4 | J | 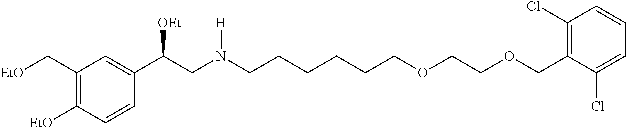 [SMILES: CCOCc1cc([C@H](CNCCCCCCOCCOCc2c(Cl)cccc2Cl)OCC)ccc1OCC] | Tri ethyl derivative of vilanterol |

In another embodiment, the present invention provides highly chiral pure oxazolidinone compound of formula IX, wherein the R-isomer is greater than 99.8% chiral pure.

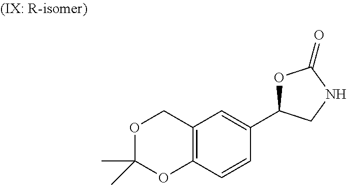 [SMILES: CC1(C)OCc2cc([C@@H]3CNC(=O)O3)ccc2O1]

In another embodiment, the present invention provides a pharmaceutical composition comprising vilanterol or a pharmaceutically acceptable salt thereof prepared by the processes of the present invention and at least one pharmaceutically acceptable excipient. Such pharmaceutical composition may be administered to a mammalian patient in any dosage form, e.g., liquid, powder, injectable solution, etc.

The present invention provides vilanterol or a pharmaceutically acceptable salt thereof and its intermediates, obtained by the above process, as analyzed using high performance liquid chromatography (“HPLC”) and Chiral HPLC with the conditions are tabulated below:

| | |
|---|---|
| Column | Inertsil ODS 3V (150 × 4.6)mm, 5 μm |
| Column/Sample | 40/25° C. |
| temperature | |
| Mobile phase | Mobile phase A: Buffer:Acetonitrile (95:5 v/v) |
| | Mobile phase B: Buffer:Acetonitrile (10:90 v/v) |
| Buffer | 0.01M KH₂PO₄ in water, pH-2.0 with H₃PO₄ |
| Diluent | Water:Acetonitrile(1:1) |
| Flow rate | 1.0 mL/min |
| Wavelength | 220 nm |
| Injection Volume | 10 μL |
| Run time | 65 min |
| Elution | gradient |
| Column | Chiralcel OJ-H (250 × 4.6) mm, 5.0 μm |
| Column/Sample | 15/25° C. |
| temperature | |
| Mobile phase | n-Hexane:EtOH:MeOH:DEA (90:05:05:0.1) |
| Diluent | n-Hexane:Ethanol (1:1) |
| Flow rate | 1.5 mL/min |
| Wavelength | 220 nm |
| Injection Volume | 20.0 μL |
| Run time | 30 min |
| Concentration | 5.0 mg/mL |
| Elution | Isocratic |

### EXAMPLES

The present invention will now be further explained in the following examples describing in detail the preparation of the said salt forms. However, the present invention should not be construed as limited thereby. One of ordinary skill in the art will understand how to vary the exemplified preparations to obtain the desired results. The reactions herein disclosed were monitored by TLC (Thin Layer Chromatography) and HPLC (High performance liquid chromatography) methods respectively.

### Example 1

### Preparation of 6-bromo-2,2-dimethyl-4H-1,3-benzodioxine (III)

5-bromo-2-hydroxy benzyl alcohol (1.0 eqt), acetone (6V) and THF (4V) were charged together at 25-30° C. under nitrogen. The reaction mass was cooled to 0-5° C. AlCl₃ (0.35eqt) was added slowly in portions to the reaction mass at 0-5° C. The reaction mass was raised to 25-30° C. and allowed to stir over a period of 1 hr. After completion of the reaction the temperature of the reaction mass lowered to 0-5° C. and quenched with 10% NaOH solution. The compound was extracted into toluene and further evaporated under vacuum below 60° C. to obtain the desired compound as brown color liquid.

Yield: 92%; purity by HPLC: 99.43%

### Example 2

### Preparation of 1-(2,2-dimethyl-4H-1,3-benzodioxin-6-yl) ethanone (IV)

Compound III (1.0eqt) was dissolved in THF (10V) at 25-30° C. and was cooled to −70 to −75° C. n-Butyl lithium (1.5 eqt) was added slowly the above reaction mass at the set temperature and stirred over a period of 60 min. N-methoxy-N-methylacetamide (1.5eqt) in THF (1V) was added to the above reaction mass at −70 to −75° C. The reaction mixture was further allowed for completion. After the completion of reaction, the temperature was raised to −30 to −20° C. and treated with 1N HCl. The reaction mass was further raised to 20-25° C. and ethyl acetate was added. The organic fractions were collected and treated with water. The combined organic fractions were dried over sodium sulfate and concentrated under vacuum to obtain the compound. This was then co distilled with heptane (5V). The crude compound was slurried with diisopropylether-heptane (1:3) at 0-10° C. The resultant was filtered and washed with heptane and suck dried under vacuum. The resultant compound was dried under vacuum over a period of 4 hr to obtain the desired compound (IV).

Yield: 53%; purity by HPLC: 99.97%

### Example 3

### Preparation of 2-bromo-1-(2,2-dimethyl-4H-1,3-benzodioxin-6-yl)ethanone (V)

Compound IV (1.0 eqt) was dissolved in THF (30V) at RT and the temperature of the reaction mass lowered to −70 to −75° C. Sodium bis(trimethylsilyl)amide (1M in THF; 1.1 eqt) was added slowly to the above reaction mass and allowed to stir over a period of one hour. TMSCl (1.05eqt) was added to the above reaction mass slowly. A solution of bromine (1.8 eqt) was added to the above and the reaction maintained at −70 to −75° C. After the completion of the reaction, the reaction mass temperature was raised to −25° C. and MTBE was added. The reaction mass was quenched with 5% sodium sulphite solution and the organic fractions were collected and treated with brine. The organic fraction was separated and dried over sodium sulfate and washed with MTBE. The MTBE fraction was evaporated under vacuum at 40-45° C. The crude product was azeotroped with heptane followed by heptane washings followed by filtration and dried under vacuum at 35-40° C. to obtain the title compound.

Yield: 55%; purity by HPLC; 95.60%

### Example 4

### Preparation of Di(tert-butyl) 2-(2,2-dimethyl-4H-1,3-benzodioxin-6-yl)-2-oxo ethylimido dicarbonate (VI)

Compound V (1.0 eqt), di-t-butyl imidino dicarboxylate (1.0 eqt) and cesium carbonate (1.2 eqt) were mixed together at 20-25° C. in acetonitrile under nitrogen over a period of 6 hr. After completion of reaction, the reaction mass was filtered off and washed with acetonitrile and concentrated under vacuum below 50° C. The residue thus obtained was dissolved in toluene and washed with water followed by brine. The organic fractions were dried over anhydrous sodium sulfate and were concentrated under vacuum below 60° C. to obtain the crude product. The crude compound was slurried with diisopropyl ether, filtered and dried under vacuum at 40-45° C. to obtain the tilted compound.

Yield: 64%; purity by HPLC: 99.57%

### Example 5

### Preparation of tert-Butyl 2-(2,2-dimethyl-4H-1,3-benzodioxin-6-yl)-2-oxoethyl carbamate (VII)

Compound VI (1.0eqt) was dissolved in dichloromethane at room temperature. The temperature of the reaction mass was lowered to 10-15° C. Trifluoroacetic acid (1.8eqt) was added slowly to the above reaction mass. The temperature of the reaction mass raised to 20-25° C. and maintained over a period of 5 h. The reaction mass was quenched with 5% aqs.NaOH. The organic fractions were separated and treated with brine, followed by separating the organic fraction and evaporating the organic fractions under vacuum, followed by treating the residue with heptane at below 50° C. to obtain the crude compound. The crude compound was slurried with mixture of diisopropyl ether-heptane. The solution was thus filtered, dried under vacuum at 40-45° C. for 4 hr period to obtain the titled compound.

Yield: 76%; purity by HPLC: 98.94%

### Example 6

### Preparation of tert-Butyl (2R)-2-(2,2-dimethyl-4H-1,3-benzodioxin-6-yl)-2-hydroxyethyl carbamate (VIII)

A 2 M solution of borane-DMS in THF (0.166 eqt) was added slowly to a 1M solution of (R)-tetrahydro-1-methyl-3,3-diphenyl-1H,3H-pyrrolo[1,2-c][1,3,2]oxazaborole in toluene (0.166 eqt) at −10° C. A solution of compound VII (1.0eqt) in THF was added slowly to the above complex at −10° C. followed by 2M solution of borane-dimethyl sulphide (1.5 eqt) in THF. The reaction mass was allowed to stir over a period of 60 min. After the completion of reaction, the reaction mass was quenched with 2M HCl (4V) under cooling and the mixture was partitioned between ethyl acetate and water. The organic fractions were treated with brine, followed by drying over sodium sulfate and concentrated under reduced pressure below 50° C. to obtain the residue. The residue was azeotroped with heptane (5V) and followed by stirring. The compound was filtered and washed with heptane and dried under vacuum at 40-45° C. The compound thus obtained found to be 86.54% pure by HPLC, with a high impurity at 0.44RRT (˜12%). The chiral purity of the compound found to be 97.05% (R-isomer) and 2.95% (S-isomer). The compound was further purified by dissolving in acetone at room temperature followed by precipitating by adding cold water at lower temperatures. The reaction mass was stirred over a period of 60 min followed by filtration and washed with chilled water, suck dried under vacuum. The product was dried under vacuum at a temperature 50-55° C. to obtain the titled compound.

Yield: 70%; purity by HPLC: 98.06%; Chiral purity: R-isomer: 97.90%; S-isomer: 2.20%

### Example 7

### Preparation of (5R)-5-(2,2-Dimethyl-4H-1,3-benzodioxin-6-yl)-1,3-oxazolidin-2-one (IX)

Compound VIII (1.0eqt) in DMF (10V) was cooled to 10-15° C., added potassium tert-butoxide (1.1 eqt) slowly in portions. The reaction mass was allowed to stir over a period of one hour at room temperature. The mixture was cooled to 10° C. and chilled water was added. This was allowed to stir over a stipulated time period and filtered, suck dried. The crude product thus obtained was dissolved in DMF (˜0.2V) and was cooled to 10-15° C. and slowly added drop wise ice cold water. The precipitated mass was allowed to stir for 1 h at 10-15° C. The reaction mass was filtered, suck dried and the product was dried under vacuum at 54-55° C. over a period of time 5-6 h, to obtain highly chiral pure titled compound.

Yield: 70%; purity by HPLC: 99.99%; Chiral purity: R-isomer: 99.97%; S-isomer: 0.03%

### Example 8

### Preparation of (2-(2,6-Dichlorobenzyloxy) ethanol (XI)

Potassium tert-butoxide (1.5eqt) was added portion wise to ethylene glycol (9.35V under nitrogen keeping the temperature below 35° C. The temperature of the reaction mass was raised to 40-45° C. and stirred for one hour period. 2,6-dichlorobenzyl bromide was charged to the above reaction mass at 40-45° C. and the mixture heated to 55-60° C. for 1 h. After completion of the reaction, cooled down to 20° C. and water was added and the mixture extracted with toluene. The aqueous layer was separated and extracted twice with toluene. The combined organic extracts were dried over sodium sulfate, filtered, and then evaporated to dryness to afford the desired compound as colorless oil.

Yield: 97.5%; purity by HPLC: 96.93%

### Example 9

### Preparation of 2-[2-(6-Bromo-hexyloxy)-ethoxymethyl]-1,3-dichloro-benzene (XII)

50% aq NaOH (4V), 2-(2,6-dichlorobenzyloxyl)ethanol (1.0eqt), 1,6-dibromohexane (5.0 eqt; less than 0.1% of 1.5-dibromo pentane) and tetrabutylammonium bromide (0.05eqt) in toluene (4V) were heated to 55-60° C. for 8-20 h. Upon completion, the reaction mass was diluted with water and toluene under cooling. The aqueous phase was separated and diluted with water then back extracted with toluene. The combined toluene extracts were washed twice with water and then evaporated to dryness under vacuum to afford the crude compound. The excess dibromo hexane was removed by vacuum distillation to get brown color oil, followed by column chromatography (Eluent: EtOAc-hexane). The pure fractions were concentrated under vacuum to afford the title compound.

Yield: 77%; purity by HPLC: 99.20%

### Example 10

### Preparation of (R)-3-{6-[2-(2,6-Dichlorobenzyloxy)-ethoxy]-hexyl}-5-(2,2-di methyl-4H-benzo[1,3]dioxin-6-yl)-oxazolidin-2-one (XIII)

Potassium tert-butoxide (1.0eqt) was added to a solution of Compound IX (1.0eqt) in DMF (7V) under N₂ and the reaction stirred for 1 h at ambient temperature. A solution of compound XII (1.0eqt) in anhydrous DMF (1.5V) was added to the above reaction mass and the reaction allowed stirring at ambient temperature for 3 hr. The reaction mixture was diluted with ice/water and extracted with ethyl acetate. The organic layer was separated then washed successively with water/saturated brine and dried over sodium sulfate. The solution was concentrated to dryness and the residue thus obtained was purified by column chromatography (EtOAc-hexane). The pure fractions were concentrated under vacuum to afford the title compound as pale yellow color oil.

Yield: 91%; purity by HPLC: 99.24%; Chiral purity: R-isomer: 99.96%; S-isomer: 0.04%

### Example 11

### Preparation of 4-(1R)-2-[(6-{2-[(2,6-Dichlorobenzyl)oxy]ethoxy}hexyl)amino]-1-(2,2-dimethyl-4H-1,3-benzodioxin-6-yl) ethanol (XIV)

Compound XIII (1.0eqt) was dissolved in THF (50V) at ambient temperature. Potassium trimethylsilanolate (4.0eqt) was added to the above reaction mass at ambient temperature under nitrogen. The temperature of the reaction mass was raised to 60-65° C. and stirred for 2 hour period. After completion of the reaction, the reaction mass was cooled to 5-10° C., treated with 5% sodium phosphate solution (pH: 6-7) and extracted with ethyl acetate. The organic layer was separated then washed successively with water/saturated brine and dried over sodium sulfate. The solution was concentrated to dryness under vacuum to obtain the residue, followed by column chromatography (MeOH-DCM). The pure fractions were concentrated under vacuum to afford the title compound as pale yellow color oil.

Yield: 85%; purity by HPLC: 98.98%; Chiral purity: R-isomer: 99.98%; S-isomer: 0.02%

### Example 12

### Preparation of 4-((R)-2-{6-[2-(2,6-Dichlorobenzyloxy)-ethoxy]-hexylamino}-1-hydroxy ethyl)-2-hydroxymethyl-phenol (I-Vilanterol)

Compound XIV (1.0 eqt) was dissolved in acetone (10V) under nitrogen at ambient temperature. The reaction mass was cooled to 0-5° C. and 0.5N HCl (12V) was added slowly. The reaction mass was allowed to stir for completion over one hour period. The reaction mass was diluted with dichloromethane and water, followed by addition of saturated sodium bicarbonate solution (10 v) at 0-5° C. The organic layer was separated then washed successively with water/saturated brine and dried over sodium sulfate the solution was concentrated to dryness under vacuum to obtain the residue, followed by column chromatography (MeOH-DCM as eluent). The pure fractions were concentrated under vacuum to afford the title compound as pale yellow color oil.

Yield: 77%; purity by HPLC: 99.15%; Chiral purity: R-isomer: 99.97%; S-isomer: 0.03%

### Example 13

### Preparation of 4-((R)-2-{6-[2-(2,6-Dichlorobenzyloxy)-ethoxy]-hexylamino}-1-hydroxy ethyl)-2-hydroxymethyl-phenol triphenyl acetate (IA: Vilanterol trifenatate)

Triphenyl acetic acid (1.0eqt) was added to a solution of compound I (1.0eqt) in acetone (20V) at ambient temperature and the mixture heated to 50-55° C. to obtain a homogenous solution. The mixture was allowed to cool to ambient temperature; the resultant product was filtered, washed with chilled acetone, dried under vacuum at 50° C. to afford the title compound as a white solid.

Yield: 69%; purity by HPLC: 99.79%; chiral purity-R-isomer: 99.96%; S-isomer: 0.049%

We claim:

1. An improved process for preparing vilanterol or a pharmaceutically acceptable salt thereof of Formula I; 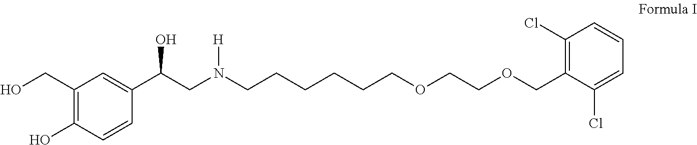 [SMILES: OCc1cc([C@@H](O)CNCCCCCCOCCOCc2c(Cl)cccc2Cl)ccc1O]
comprising:
a) reacting 2,6-dichlorobenzyl halide 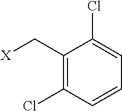
(X is either F or Cl or Br or I);
with ethylene glycol in presence of a suitable base to obtain 2-(2,6-dichlorobenzyloxy) ethanol of 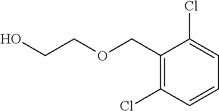 [SMILES: OCCOCc1c(Cl)cccc1Cl]
b) reacting the 2-(2,6-dichlorobenzyloxy) ethanol with 1,6-dihalo hexane of formula 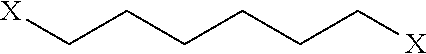
(X is either F or Cl or Br or I);
in presence of a base and optionally in presence of a phase transfer catalyst in a suitable organic solvent to obtain 2-[2-(6-halo-hexyloxy)-ethoxymethyl]-1,3-dichloro-benzene Formula XIIA, 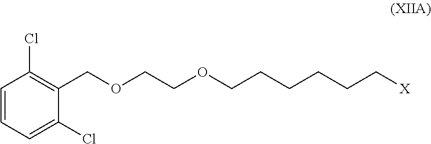
(X is either F or Cl or Br or I)
c) condensing the resultant compound of Formula XIIA with (5R)-5-(2,2-dimethyl-4H-1,3-benzodioxin-6-yl)-1,3-oxazolidin-2-one of Formula IX, 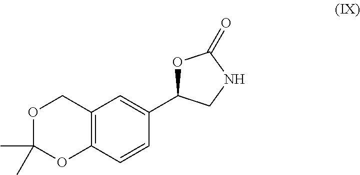 [SMILES: CC1(C)OCc2cc([C@@H]3CNC(=O)O3)ccc2O1]
in presence of a suitable base and a solvent to obtain (5R)-3-{6-[2-(2,6-dichloro benzyloxy)-ethoxy]-hexyl}-5-(2,2-dimethyl-4H-benzo[1,3]dioxin-6-yl)-oxazolidin-2-one 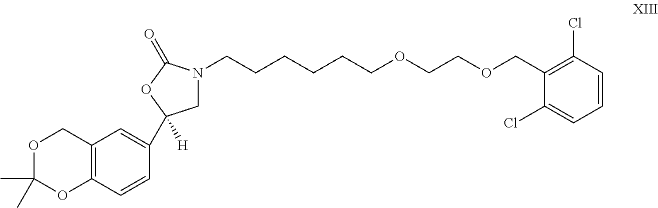 [SMILES: CC1(C)OCc2cc([C@@H]3CN(CCCCCCOCCOCc4c(Cl)cccc4Cl)C(=O)O3)ccc2O1]
d) cleaving the compound of step (c) in presence of a suitable base and a suitable solvent to obtain 4-(1R)-2-[(6-{2-[(2,6-dichlorobenzyl)oxy]ethoxy}hexyl)amino]-1-(2,2-di methyl-4H-1,3-benzodioxin-6-yl) ethanol of Formula XIV, 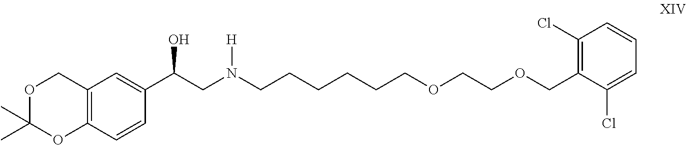 [SMILES: CC1(C)OCc2cc([C@@H](O)CNCCCCCCOCCOCc3c(Cl)cccc3Cl)ccc2O1]
e) deprotecting the compound of step (d) in presence of an acid and a suitable nonalcoholic solvent to obtain substantially pure vilanterol,
f) optionally converting vilanterol into a pharmaceutically acceptable salt of vilanterol in a suitable nonalcoholic solvent medium.

2. The process of claim 1, wherein the ‘X’ is bromo.

3. The process of claim 1, wherein the base in step a) is potassium t-butoxide.

4. The process of claim 1, wherein the 1,6-dihalo hexane contain less than 0.15% of corresponding dihalo alkane derivatives.

5. The process of claim 4, wherein the dihaloalkane derivative is dibromopentane.

6. The process of claim 1, wherein the base in step b) is lithium hydroxide, sodium hydroxide or potassium hydroxide.

7. The process of claim 1, wherein the phase transfer catalyst is selected from the group comprising tetra butyl ammonium chloride, tetra butyl ammonium bromide or tetra butyl ammonium iodide.

8. The process of claim 1, wherein the suitable base in step c) is potassium t-butoxide.

9. The process of claim 1, wherein the solvent in step c) is selected from dimethyl formamide (DMF), dimethyl acetamide (DMAc), dimethyl sulfoxide (DMSO), n-methyl-pyrrolidin-2-one (NMP) and mixtures thereof.

10. The process of claim 1, wherein the base in step d) is potassium trimethyl silanolate and the solvent is THF.

11. The process of claim 1, wherein the nonalcoholic solvent is selected from the group consisting of halogenated solvents, esters, ethers, hydrocarbons, amides, ketones, nitriles and mixtures thereof.

12. The process of claim 11, wherein the nonalcoholic solvent is acetone.

13. The process of claim 1, wherein the acid in step e) is selected from the group consisting of HCl, HBr, DMF-HCl, EtOAc—HCl, ether-HCl, dioxane-HCl.

14. The process of claim 13, wherein the acid is HCl.

15. The process of claim 1, wherein the pharmaceutically acceptable salt is triphenyl acetic acid.

16. An improved process for preparing vilanterol or a pharmaceutically acceptable salt thereof, comprising:
a) deprotecting 4-(1R)-2-[(6-{2-[(2,6-dichloro benzyl)oxy]ethoxy}hexyl)amino]-1-(2,2-di methyl-4H-1,3-benzodioxin-6-yl)ethanol of Formula XIV 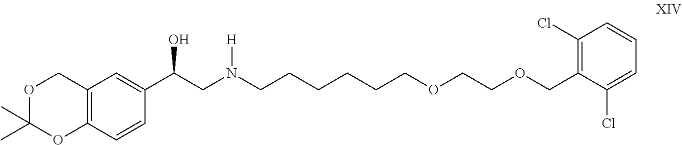 [SMILES: CC1(C)OCc2cc([C@@H](O)CNCCCCCCOCCOCc3c(Cl)cccc3Cl)ccc2O1]
with a suitable acid in presence of a nonalcoholic solvents to obtain vilanterol, and
b) optionally converting the vilanterol into a pharmaceutically acceptable salt of vilanterol in a suitable nonalcoholic solvent medium.

17. The process of claim 16, wherein the nonalcoholic solvent is selected from the group consisting of halogenated solvents, esters, ethers, hydrocarbons, amides, ketones, nitriles and mixtures thereof.

18. The process of claim 17, wherein the nonalcoholic solvent is acetone.

19. The process of claim 16, wherein the acid in step e) is selected from the group consisting of HCl, HBr, DMF-HCl, EtOAc—HCl, ether-HCl, dioxane-HCl.

20. The process of claim 19, wherein the acid is HCl.

21. An improved process for preparing vilanterol or a pharmaceutically acceptable salt thereof, comprising preparing substantially pure chiral oxazolidinone intermediate of Formula IX, 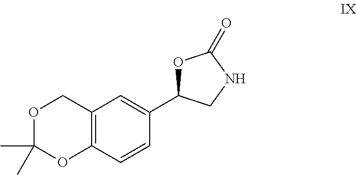 [SMILES: CC1(C)OCc2cc([C@@H]3CNC(=O)O3)ccc2O1]
comprising:
i) reaction of 2-bromo-1-(2,2-dimethyl-4H-1,3-benzodioxin-6-yl) ethanone of Formula V with di-tert-butyl iminodicarboxylate 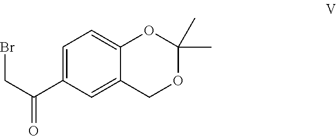 [SMILES: CC1(C)OCc2cc(C(=O)CBr)ccc2O1]
in the presence of a suitable base and an aprotic solvent to obtain di(tert-butyl) 2-(2,2-dimethyl-4H-1,3-benzodioxin-6-yl)-2-oxoethyl imidodicarbonate of Formula VI, 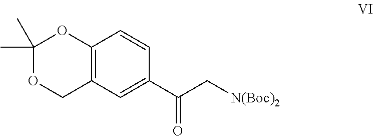
ii) deprotecting the resultant compound from step (i) in the presence of acid to obtain tert-butyl 2-(2,2-dimethyl-4H-1,3-benzodioxin-6-yl)-2-oxoethylcarbamate of Formula VII, 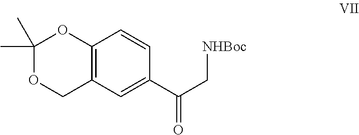 [SMILES: CC(C)(C)OC(=O)NCC(=O)c1ccc2c(c1)COC(C)(C)O2]
iii) reducing the compound of step (ii) with a suitable reducing agent in the presence of suitable chiral auxiliary to obtain substantially pure tert-butyl (2R)-2-(2,2-dimethyl-4H-1,3-benzodioxin-6-yl)-2-hydroxyethyl carbamate of Formula VIII, 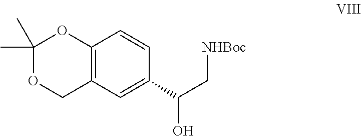
iv) cyclisation of compound of step (iii) in presence of a suitable base and a solvent to obtain substantially pure (5R)-5-(2,2-Dimethyl-4H-1,3-benzodioxin-6-yl)-1,3-oxazolidin-2-one, and
v) conversion of substantially pure (5R)-5-(2,2-Dimethyl-4H-1,3-benzodioxin-6-yl)-1,3-oxazolidin-2-one to vilanterol or pharmaceutically acceptable salts thereof.

22. The process of claim 21, wherein the suitable base in step (i) is selected from sodium carbonate, potassium carbonate or cesium carbonate.

23. The process of claim 21, wherein the aprotic solvent is step (i) is selected from the group consisting of acetonitrile, tetrahydrofuran, dioxane, DMF, DMAC, DMSO, NMP and mixtures thereof.

24. The process of claim 21, wherein the suitable base is cesium carbonate and the aprotic solvent is acetonitrile.

25. The process of claim 21, wherein the acid in step (ii) is selected from HCl, HBr or trifluoroacetic acid.

26. The process of claim 21, wherein the tert-butyl 2-(2,2-dimethyl-4H-1,3-benzodioxin-6-yl)-2-oxoethylcarbamate of Formula VII is isolated by slurring the crude compound in organic solvent.

27. The process of claim 26, wherein the organic solvent is selected from the group consisting of diethyl ether, diisopropylether, hexane, heptane and mixtures thereof.

28. The process of claim 27, wherein the organic solvent is mixture of diisopropylether and heptane.

29. The process of claim 21, wherein the suitable reducing agent is borane-DMS and the suitable chiral auxiliary is (R)-MeCBS.

30. The process of claim 21, further comprises purifying the tert-butyl (2R)-2-(2,2-dimethyl-4H-1,3-benzodioxin-6-yl)-2-hydroxyethyl carbamate of Formula VIII by:
a) dissolving tert-butyl (2R)-2-(2,2-dimethyl-4H-1,3-benzodioxin-6-yl)-2-hydroxyethyl carbamate of Formula VIII in a suitable organic solvent,
b) adding an anti solvent to step a) solution,
c) isolating the tert-butyl (2R)-2-(2,2-dimethyl-4H-1,3-benzodioxin-6-yl)-2-hydroxyethyl carbamate of Formula VIII.

31. The process of claim 30, wherein the suitable organic solvent is acetone or methyl isobutyl ketone.

32. The process of claim 30, wherein the anti solvent is selected from the group consisting of pentane, hexane, heptane, toluene, xylene, dimethyl ether, diethyl ether, diisopropyl ether, Methyl tertiary butyl ether, water or mixtures thereof.

33. The process of claim 30, wherein the suitable organic solvent is acetone and the anti solvent is water.

34. The process of claim 21, wherein the suitable base in step (iv) is selected from alkali metal alkoxides, alkali metal hydroxides, alkali metal carbonates or alkali metal bicarbonates.

35. The process of claim 34, wherein the suitable base is potassium tert-butoxide.

36. The process of claim 21, wherein the solvent in the step (iv) is dimethyl formamide.

37. A process for purification of (5R)-5-(2,2-dimethyl-4H-1,3-benzodioxin-6-yl)-1,3-oxazolidin-2-one of formula IX, comprising:
a) dissolving the crude compound in a suitable solvent
b) adding suitable anti solvent to precipitation,
c) filtering to obtain substantially pure desired isomer.

38. The process of claim 37, wherein the suitable solvent is polar aprotic solvent.

39. The process of claim 38, wherein the suitable solvent is dimethyl formamide.

40. The process of claim 37: wherein the anti solvent is water.

41. (5R)-5-(2,2-Dimethyl-4H-1,3-benzodioxin-6-yl)-1,3-oxazolidin-2-one of Formula IX having less than 0.2% of its corresponding (S)-isomer as measured by HPLC.

42. Vilanterol or a pharmaceutically acceptable salt thereof having a total purity greater than 98% as measured by HPLC.

43. Vilanterol or a pharmaceutically acceptable salt thereof having a chiral purity greater than 98% as measured by HPLC.

44. Vilanterol or a pharmaceutically acceptable salt thereof is having less than 0.2% of its corresponding (S)-isomer as measured by HPLC.

45. Vilanterol or a pharmaceutically acceptable salt thereof containing less than 0.15% as measured by HPLC of one or more impurities. 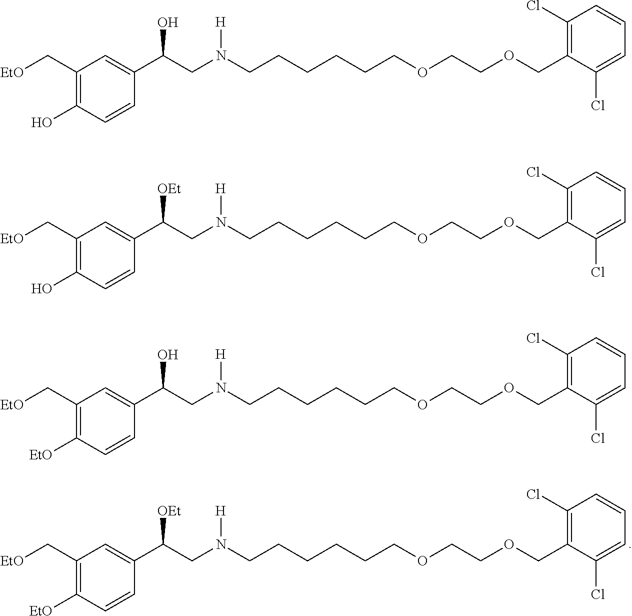 [SMILES: CCOCc1cc([C@@H](O)CNCCCCCCOCCOCc2c(Cl)cccc2Cl)ccc1O ; CCOCc1cc([C@H](CNCCCCCCOCCOCc2c(Cl)cccc2Cl)OCC)ccc1O ; CCOCc1cc([C@@H](O)CNCCCCCCOCCOCc2c(Cl)cccc2Cl)ccc1OCC ; CCOCc1cc([C@H](CNCCCCCCOCCOCc2c(Cl)cccc2Cl)OCC)ccc1OCC]
